(19) United States
(12) Patent Application Publication
(10) Pub. No.: US 2011/0110925 A1
(43) Pub. Date: May 12, 2011

(54) Compositions and Methods for the Treatment and Diagnosis of Cancer

(75) Inventors: David L. Wiest, Doylestown, PA (US); Thomas A. Look, North Reading, MA (US)

(73) Assignees: Fox Chase Cancer Center, Philadelphia, PA (US); Dana Farber Cancer Institute, Boston, MA (US)

(21) Appl. No.: 12/950,532

(22) Filed: Nov. 19, 2010

Related U.S. Application Data
(63) Continuation-in-part of application No. PCT/US09/44669, filed on May 20, 2009.
(60) Provisional application No. 61/054,662, filed on May 20, 2008.
(60) Provisional application No. 61/150,996, filed on Feb. 9, 2009.

(51) Int. Cl. A61K 39/395; C12Q 1/68; C12Q 1/02; A61K 31/7088; A61K 38/02; A61P 35/00; A61P 35/02

(52) U.S. Cl. 424/130.1; 435/6; 435/29; 514/44 R; 514/19.3

(57) ABSTRACT

Compositions and methods for the diagnosis and treatment of cancer, particularly T-ALL, are disclosed.

This application is a §365 application of PCT/US2009/44669 filed May 20, 2009 which claims priority to U.S. Provisional Application 61/054,662 filed May 20, 2008 and U.S. Provisional Application 61/150,996 filed Feb. 9, 2009, the entire disclosures of each being incorporated by reference herein as though set forth in full.

### FIELD OF THE INVENTION

This invention relates to the fields of oncology and hematology. More specifically, the invention provides compositions and methods for the diagnosis and treatment of cancer, particularly cancers which arise from cells of hematopoietic lineage.

### BACKGROUND OF THE INVENTION

Several publications and patent documents are cited throughout the specification in order to describe the state of the art to which this invention pertains. Each of these citations is incorporated by reference herein as though set forth in full.

T cells mature in the thymus through a well-defined series of stages that can be delineated by changes in expression of the co-receptors CD4 and CD8. The least mature T cell precursors enter the thymus as CD4⁻CD8⁻ (double negative or DN) thymocytes, which progress to the CD4⁺CD8⁺ (double positive or DP) stage before selectively silencing one of these co-receptors to become mature CD4⁺ or CD8⁺ single positive (SP) cells. During thymopoiesis, two major types of mature T cells are generated that can be distinguished by the clonotypic subunits contained within their T cell receptor complexes (TCR): αβ T cells and γδ T cells. See FIG. 1. These two lineages are thought to derive from a common DN precursor (Petrie et al., (1992) European Journal of Immunology 22, 2185-2188) and to separate from one another between the CD44⁺CD25⁺ (DN2) and CD44⁻CD25⁺ (DN3) stages of development (Ciofani et al., (2006) Immunity 25, 105-116). DN thymocytes that productively rearrange the T cell receptor (TCR) γ and TCRδ loci and express a mature γδTCR (TCRγδ heterodimer associated with CD3γε and ζ) are capable of differentiating along the γδ lineage pathway while remaining DN (Kang et al., (1998) Immunity 8, 427-438; Passoni et al., (1997) Immunity 7, 83-95). In contrast, cells that bear an in-frame TCRβ rearrangement express the pre-TCR (TCRβ-pTα heterodimer associated with CD3γδε and ζ) and are able to differentiate along the αβ lineage pathway to the DP stage. Precursors that have matured to the DP stage in response to pre-TCR signals are said to have been β-selected. β-selection stipulates that only those DN3 thymocytes in which V(D)J recombination produces a functional TCRβ protein will survive and differentiate; those failing to do so die by apoptosis (Dudley et al., (1994) Immunity 7, 83-95; Hoffman et al., (1996) Genes & Development 10, 948-962. Pre-TCR signals that induce traversal of the β-selection checkpoint produce 4 developmental outcomes: 1) rescue of those DN3 thymocytes from apoptosis; 2) extensive proliferative expansion (Hoffman et al., 1996, supra); 3) allelic exclusion at the TCRβ locus, i.e., termination of V(D)J recombination at the remaining β allele (Aifantis et al., (1997) Immunity 7, 601-607; and 4) differentiation to the DP stage (Kruisbeek et al., (2000) Immunology Today 21, 637-644.

While signaling through the pre-TCR and γδTCR complexes facilitates development of αβ and γδ lineage precursors, respectively, the precise role that these receptors play in selection of the αβ and γδ lineages has been controversial (reviewed in (Hayes et al., (2003) Immunol Rev 191, 28-37; MacDonald and Wilson, (1998) Immunological Reviews 165, 87-94). We and others have recently provided support for the signal strength model of αβ vs. γδ lineage commitment which posits that strong signals direct development to the γδ lineage whereas relatively weaker signals promote commitment to the αβ lineage (Haks et al., (2005) Immunity 22, 595-606; Hayes et al., (2005) Immunity 22, 583-593. In particular, we showed that differential activation of the extracellular signal-regulated kinase (ERK)-early growth response (Egr)-inhibitor of differentiation 3 (Id3) pathway is an important element of the TCR signals that regulate αβ vs. γδ lineage choice and development (Haks et al., (2005, supra). Nevertheless, the regulatory cascades that control γδ lineage commitment and development, and how these differ from those involved in adoption of the αβ fate, remain to be established.

Rpl22 is a component of the 60S large ribosomal subunit and co-localizes with ribosomal RNA in the nucleolus and the cytoplasm (Lavergne et al., (1987) FEBS Lett 216, 83-88; Shu-Nu et al., (2000) FEBS Lett 484, 22-28; Toczyski et al., (1994) Proc Natl Acad Sci USA 91, 3463-3467). Although ubiquitously expressed and associated with the ribosome, Rpl22 is not required for translation in vitro (Lavergne et al., (1987, supra). Nevertheless, it remains possible that Rpl22 may play a cell type- or developmental stage-specific role in protein synthesis or ribosome assembly. It has also been suggested that Rpl22 may play a role in assembly or function of other multi-subunit ribonucleoprotein (RNP) particles. Accordingly, Rpl22 has been found to associate with both viral RNAs and proteins in infected cells and to be a component of the telomerase holoenzyme complex. However, to date Rpl22 has not been implicated in any specific developmental pathway, mechanism, or disease.

While ribosomal proteins are increasingly understood to be important focuses of cellular regulation and transformation, little is known about the molecular basis by which mutations in particular ribosomal proteins contribute to the etiology of cancer. It is an object of the present invention to elucidate these underlying molecular mechanisms, thereby identifying new targets to facilitate the characterization of new anti-proliferative agents and methods of use thereof for the diagnosis and treatment of cancer.

### SUMMARY OF THE INVENTION

In accordance with the present invention, compositions and methods are provided for diagnosis and treatment of cancer, particularly, T-ALL An exemplary method entails detecting the presence of a mutation in a Rpl22 coding sequence in a target polynucleotide wherein if the mutation is present, the patient has an increased risk for developing cancer.

The methods of the invention can include alternative means for detecting the disclosed mutations and deletions. For example, such methods of detection can further comprises processes such as specific hybridization, measurement of allele size, restriction fragment length polymorphism analysis, allele-specific hybridization analysis, single base primer extension reaction, and sequencing of an amplified polynucleotide.

The invention also encompasses screening methods to identify agents which modulate or can substitute for Rpl22 function. An exemplary method entails providing cells comprising at least one mutation in Rpl22; providing cells which lack the cognate mutation; contacting each cell type with a test agent and analyzing whether said agent alters an Rpl22 associated parameter. Agents so identified are also within the scope of the invention.

Also provided are mice comprising non-functional Rpl22. Such mice provide a superior in vivo screening tool to identify agents which modulate the progression and development of cancer. In a preferred embodiment Rpl22 in the gene-targeted mice of the invention is inactivated via insertional mutagenesis.

### BRIEF DESCRIPTION OF THE DRAWINGS

FIG. 1 is a schematic diagram of thymocyte development. Black oval denotes proliferation

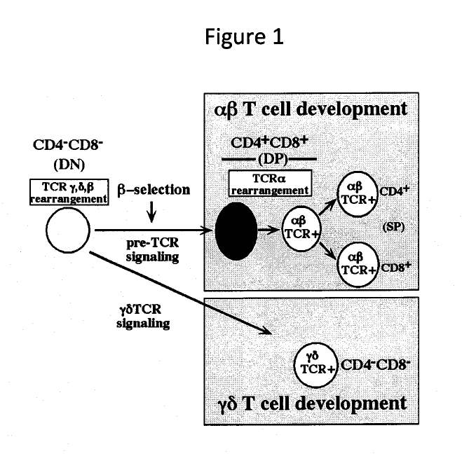

FIG. 2 is a Western blot and a series of histograms showing Rpl22-deficiency induces p53 in DN3 thymocytes and arrests T cell development. A. Blots on isolated thymocyte subpopulations. B. Flow cytometry on thymocyte suspensions.

FIG. 3 is a graph showing Rpl22+/− mice expressing a MyrAkt2 Tg develop thymic lymphoma more rapidly that Rpl22+/+ mice. Mice of the indicated genotypes were sacrificed upon showing evidence of disease. All mice that were sacrificed had thymic lymphoma.

FIG. 4 shows aCGH analysis on primary T-ALL samples. DNA from primary T-ALL samples was subjected to copy number analysis using Agilent arrays. Vertical dark blue bars denote the position of the deletion.

FIG. 5 shows the results of sequencing data which reveals a common single nucleotide deletion produces a frameshift mutation in RPL22 in T-ALL cell lines. Genomic DNA from a panel of 19 T-ALL cell lines was sequenced using 4 amplicons spanning the coding regions of the RPL22 gene. Arrow indicates position of monoallelic loss of a single A.

| WT Rpl22-N-term: | |
|---|---|
| MAPVKKLVVKGGKKKKQV . . . ; | (SEQ ID NO 1) |
| Mut Rpl22-N-term: | |
| MAPVKKLVVKGGKKRSKF*. | (SEQ ID NO: 2) |

FIG. 6 depicts gels showing that Rpl22 binds to p53 mRNA and regulates its translation in vitro. FIG. 6A: Extracts of thymocytes were immunoprecipiated using the indicated Ab, following which bound RNA was extracted and amplified by RT-PCR. FIG. 6B: In vitro transcribed p53 mRNA was translated in vitro in reactions supplemented with the indicated fusion proteins. Radiolabeled proteins were resolved by SDS-PAGE and visualized by phosphorimagery.

FIG. 7 provides data showing the effect of Rpl22-deficiency on p27Kip protein expression and proliferation. FIG. 7A: Detergent extract of Splenic T cells from Rpl22+/+ and Rpl22−/− mice that were either untreated or induced to proliferate by anti-CD3 stimulation were blotted with anti-p27 or anti-calnexin A. FIG. 7B: Following a 4 h pulse of BrDU in vivo, thymic subsets from Rpl22+/+ and Rpl22−/− mice were stained with anti-BrDU Ab as well as the indicated Ab. Gate frequencies are indicated on the resulting histrograms. The increase in BrdU incorporation in DN2 and DN3 from Rpl22−/− mice relative to that in Rpl22+/+ mice is statistically significant (p<0.05).

FIG. 8 provides data showing that Rpl22 deficiency promotes transformation in acute assays of transformation in vitro.

FIG. 9 demonstrates that Rpl22 mutation induces expression of the stem cell inducing gene Lin28.

FIG. 10 is a graph showing that loss of Rpl22 is associated with an increase in Lin28B mRNA levels.

FIG. 11 provides data that show that Rpl22 deficiency increases expression of Rpl22-Like 1 which in turn stimulates cellular proliferation.

FIG. 12 provides a schematic model of Rpl22 function in transformation.

### DETAILED DESCRIPTION OF THE INVENTION

Acute lymphoblastic leukemia (ALL) is the most common pediatric cancer. Although the treatment outcome in ALL patients in which T lymphocytes have become transformed (T-ALL) has improved in recent years, patients with relapsed disease continue to have a dismal prognosis, despite the advent of hematopoietic stem cell transplantation. It is thus very important to investigate the molecular pathways that control both transformation and the acquisition of treatment resistance in T-ALL. The early success of molecularly targeted therapy in chronic myelogenous leukemia (i.e., imatinib mesylate inhibition of the BCR-ABL fusion protein), stimulated enormous interest in targeting abnormal signaling pathways in cancer cells (R. M. Mesters, T. Padro, R. Bieker et al., Blood 98 (1), 241 (2001). Despite the fact that activating Notch mutations are common in T-ALL (A. P. Weng, A. A. Ferrando, W. Lee et al., Science 306 (5694), 269 (2004), attempts to treat T-ALL with gamma secretase inhibitors (GSI; block cleavage-mediated Notch activation) have been disappointing in that many cell lines exhibit resistance (J. O'Neil, J. Grim, P. Strack et al., J Exp Med 204 (8), 1813 (2007); B. J. Thompson, S. Buonamici, M. L. Sulis et al., J Exp Med 204 (8), 1825 (2007). Recent evidence suggests that this may reflect mutations that regulate Notch activity downstream of the gamma secretase cleavage step (e.g., FBW7 which regulates Notch turnorver). An additional mechanism underlying GSI-resistance is loss of the tumor suppressor PTEN (T. Palomero, M. L. Sulis, M. Cortina et al., Nat Med 13 (10), 1203 (2007). PTEN loss activates Akt and ultimately mTOR, which produces a variety of trophic effects on cell metabolism. Accordingly, mTOR may serve as a common node of both the Notch and Akt pathways that could be exploited therapeutically. Preliminary tests in cell lines suggest this may be a fruitful approach (S. M. Chan, A. P. Weng, R. Tibshirani et al., Blood 110 (1), 278 (2007). The Myc oncoprotein is also potentially a common target of both the Notch and Akt pathways. Indeed, Notch signals directly activate Myc expression and evidence shows that Myc becomes activated through a chromosomal translocation (14:15, data not shown) in the Myr-Akt2 Tg mouse model of T-ALL. Myc overexpression has been shown to render cells resistant to GSI.

Several cancer prone syndromes are associated with mutations in ribosomal protein genes (e.g. Rps19 in Diamond Blackfan Anemia and Rps14 in 5q-Syndrome) (J. M. Lipton, Semin Hematol 43 (3), 167 (2006); B. L. Ebert, J. Pretz, J. Bosco et al., Nature 451 (7176), 335 (2008). Likewise, loss of one allele of a number of ribosomal genes led to accelerated and more penetrant development of cancer in zebrafish. Interestingly, the remaining allele of the ribosomal gene in the fish tumors was in wild type configuration, suggesting that the ribosomal proteins function as haploinsufficient tumor suppressors. These findings have not been extended to a mammalian model. Rpl22 was not identified among these genes, presumably because the screen was restricted to genes that are lethal upon homozygous disruption and Rpl22 disruption is not likely to be lethal in fish, since it is not lethal in mice. The mechanistic basis by which loss of an allele of a ribosomal protein gene promotes transformation has not been addressed. Interestingly, the ribosomal filter hypothesis suggests that ribosomes of differing subunit composition have the capacity to regulate the proteome through selective translation. While there has been little experimental support for this model, we have demonstrated that Rpl22 is capable of regulating the translation of p53 mRNA in a cell type specific manner. Moreover, our data indicates that loss of one allele of Rpl22 predisposes T lineage precursors to transformation in in vivo models and that one copy of the Rpl22 gene is inactivated in some T-ALL. Taken together, these data imply that particular ribosomal protein components may perform stage or cell type specific functions that regulate both normal development and transformation. Thus, in accordance with the present invention, we have determined that Rpl22 is part of a signaling pathway that critically regulates T cell development and transformation.

### I. DEFINITIONS

A “pharmaceutical candidate” or “drug candidate” is a compound believed to have therapeutic potential, that is to be tested for efficacy.

The “screening” of a pharmaceutical candidate refers to conducting an assay that is capable of evaluating the efficacy and/or specificity of the candidate. In this context, “efficacy” refers to the ability of the candidate to effect the cell or organism it is administered to in a beneficial way: for example, the limitation of the pathology of cancerous cells.

A “cell line” or “cell culture” denotes higher eukaryotic cells grown or maintained in vitro. It is understood that the descendants of a cell may not be completely identical (either morphologically, genotypically, or phenotypically) to the parent cell. Cells described as “uncultured” are obtained directly from a living organism, and have been maintained for a limited amount of time away from the organism: not long enough or under conditions for the cells to undergo substantial replication.

A “host cell” is a cell which has been transformed, or is capable of being transformed, by administration of an exogenous polynucleotide.

The terms “cancerous cell” or “cancer cell”, used either in the singular or plural form, refer to cells that have undergone a malignant transformation that makes them pathological to the host organism. Malignant transformation is a single- or multi-step process, which involves in part an alteration in the genetic makeup of the cell and/or the expression profile. Malignant transformation may occur either spontaneously, or via an event or combination of events such as drug or chemical treatment, radiation, fusion with other cells, viral infection, or activation or inactivation of particular genes. Malignant transformation may occur in vivo or in vitro, and can if necessary be experimentally induced.

A frequent feature of cancer cells is the tendency to grow in a manner that is uncontrollable by the host, but the pathology associated with a particular cancer cell may take another form. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells.

The “pathology” caused by a cancer cell within a host is anything that compromises the well-being or normal physiology of the host. This may involve (but is not limited to) abnormal or uncontrollable growth of the cell, metastasis, release of cytokines or other secretory products at an inappropriate level, manifestation of a function inappropriate for its physiological milieu, interference with the normal function of neighboring cells, aggravation or suppression of an inflammatory or immunological response, or the harboring of undesirable chemical agents or invasive organisms.

“Treatment” of an individual or a cell is any type of intervention in an attempt to alter the natural course of the individual or cell. For example, treatment of an individual may be undertaken to decrease or limit the pathology caused by a cancer cell harbored in the individual. Treatment includes (but is not limited to) administration of a composition, such as a pharmaceutical composition, and may be performed either prophylactically, or subsequent to the initiation of a pathologic event or contact with an etiologic agent.

A “control cell” is an alternative source of cells or an alternative cell line used in an experiment for comparison purposes. Where the purpose of the experiment is to establish a base line for gene copy number or expression level, it is generally preferable to use a control cell that is not a cancer cell.

It is understood that a “clinical sample” encompasses a variety of sample types obtained from a subject and useful in an in vitro procedure, such as a diagnostic test. The definition encompasses solid tissue samples obtained as a surgical removal, a pathology specimen, or a biopsy specimen, tissue cultures or cells derived therefrom and the progeny thereof, and sections or smears prepared from any of these sources. Non-limiting examples are samples obtained from breast tissue, lymph nodes, and tumors. The definition also encompasses blood, spinal fluid, and other liquid sample of biologic origin, and may refer to either the cells or cell fragments suspended therein, or to the liquid medium and its solutes.

The term “relative amount” is used where a comparison is made between a test measurement and a control measurement. Thus, the relative amount of a reagent forming a complex in a reaction is the amount reacting with a test specimen, compared with the amount reacting with a control specimen. The control specimen may be run separately in the same assay, or it may be part of the same sample (for example, normal tissue surrounding a malignant area in a tissue section).

A “differential” result is generally obtained from an assay in which a comparison is made between the findings of two different assay samples, such as a cancerous cell line and a control cell line. Thus, for example, “differential expression” is observed when the level of expression of a particular gene is higher in one cell than another. “Differential display” refers to a display of a component, particularly RNA, from different cells to determine if there is a difference in the level of the component amongst different cells. Differential display of RNA is conducted, for example, by selective production and display of cDNA corresponding thereto.

The practice of the present invention will employ, unless otherwise indicated, conventional techniques of molecular biology, microbiology, recombinant DNA, and immunology, which are within the skill of the art. Such techniques are explained fully in the literature. See, for example, “Molecular Cloning: A Laboratory Manual”, Second Edition (Sambrook, Fritsch & Maniatis, 1989), “Oligonucleotide Synthesis” (M. J. Gait, ed., 1984), “Animal Cell Culture” (R. I. Freshney, ed., 1987); the series “Methods in Enzymology” (Academic Press, Inc.); “Handbook of Experimental Immunology” (D. M. Weir & C. C. Blackwell, Eds.), “Gene Transfer Vectors for Mammalian Cells” (J. M. Miller & M. P. Calos, eds., 1987), “Current Protocols in Molecular Biology” (F. M. Ausubel et al., eds., 1987); and “Current Protocols in Immunology” (J. E. Coligan et al., eds., 1991).

“Pharmaceutically acceptable” indicates approval by a regulatory agency of the Federal government or a state government. “Pharmaceutically acceptable” agents may be listed in the U.S. Pharmacopeia or other generally recognized pharmacopeia for use in animals, and more particularly in humans.

A “carrier” refers to, for example, a diluent, adjuvant, excipient, auxilliary agent or vehicle with which an active agent of the present invention is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin.

A “replicon” is any genetic element, for example, a plasmid, cosmid, bacmid, plastid, phage or virus, which is capable of replication largely under its own control. A replicon may be either RNA or DNA and may be single or double stranded. Generally, a “viral replicon” is a replicon which contains the complete genome of the virus. A “sub-genomic replicon” refers to a viral replicon that contains something less than the full viral genome, but is still capable of replicating itself. For example, a sub-genomic replicon may contain most of the genes encoding for the non-structural proteins of the virus, but not most of the genes encoding for the structural proteins.

A “vector” is a replicon, such as a plasmid, cosmid, bacmid, phage or virus, to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The terms “percent similarity,” “percent identity” and “percent homology,” when referring to a particular sequence, are used as set forth in the University of Wisconsin GCG software program.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

The term “oligonucleotides” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

Polymerase chain reaction (PCR) has been described in U.S. Pat. Nos. 4,683,195, 4,800,195, and 4,965,188, the entire disclosures of which are incorporated by reference herein.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., 1989, Molecular Cloning: A Laboratory Manual, Cold Spring Harbor Laboratory Press):

Tₘ=81.5° C.+16.6 Log [Na+]+0.41(% G+C)−0.63(% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as, to exist in “substantially pure” form. “Isolated” is not meant to exclude artificial or synthetic mixtures with other compounds or materials, or the presence of impurities that do not interfere with the fundamental activity, and that may be present, for example, due to incomplete purification, or the addition of stabilizers.

The term “gene” refers to a nucleic acid comprising an open reading frame encoding a polypeptide, including both exon and (optionally) intron sequences. The nucleic acid may also optionally include non-coding sequences such as promoter or enhancer sequences. The term “intron” refers to a DNA sequence present in a given gene that is not translated into protein, is generally found between exons, and is “spliced out” during processing of the mRNA transcript. As used herein, the term “exon” refers to a nucleic acid sequence found in genomic DNA that is predicted and/or experimentally confirmed to contribute contiguous sequence to a mature (e.g., spliced) mRNA transcript and/or is translated into protein.

As used herein, the phrase “splice variants” refers to RNA molecules initially transcribed from the same genomic DNA sequence but which have undergone alternative RNA splicing. Alternative RNA splicing occurs when a primary RNA transcript undergoes splicing, generally for the removal of introns, which results in the production of more than one mRNA molecule, which may encode different amino acid sequences. The term splice variant may also refer to the proteins encoded by the above RNA molecules. As used herein, the phrase “alternative splicing” includes all types of RNA processing that lead to expression of plural protein isoforms from a single gene. As such, the phrase “splice variant” embraces mRNAs transcribed from a given gene that, however processed, collectively encode plural protein isoforms. For example, and by way of illustration only, splice variants can include exon insertions, exon extensions, exon truncations, exon deletions, alternatives in the 5′ untranslated region and alternatives in the 3′ untranslated region.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the basic and novel characteristics of the sequence.

The term “promoters” or “promoter” as used herein can refer to a DNA sequence that is located adjacent to a DNA sequence that encodes a recombinant product. A promoter is preferably linked operatively to an adjacent DNA sequence. A promoter typically increases an amount of recombinant product expressed from a DNA sequence as compared to an amount of the expressed recombinant product when no promoter exists. A promoter from one organism can be utilized to enhance recombinant product expression from a DNA sequence that originates from another organism. For example, a vertebrate promoter may be used for the expression of jellyfish GFP in vertebrates. In addition, one promoter element can increase an amount of recombinant products expressed for multiple DNA sequences attached in tandem. Hence, one promoter element can enhance the expression of one or more recombinant products. Multiple promoter elements are well-known to persons of ordinary skill in the art.

The term “enhancers” or “enhancer” as used herein can refer to a DNA sequence that is located adjacent to the DNA sequence that encodes a recombinant product. Enhancer elements are typically located upstream of a promoter element or can be located downstream of or within a coding DNA sequence (e.g., a DNA sequence transcribed or translated into a recombinant product or products). Hence, an enhancer element can be located 100 base pairs, 200 base pairs, or 300 or more base pairs upstream or downstream of a DNA sequence that encodes recombinant product. Enhancer elements can increase an amount of recombinant product expressed from a DNA sequence above increased expression afforded by a promoter element. Multiple enhancer elements are readily available to persons of ordinary skill in the art.

The terms “transfected” and “transfection” as used herein refer to methods of delivering exogenous DNA into a cell. These methods involve a variety of techniques, such as treating cells with high concentrations of salt, an electric field, liposomes, polycationic micelles, or detergent, to render a host cell outer membrane or wall permeable to nucleic acid molecules of interest. These specified methods are not limiting and the invention relates to any transformation technique well known to a person of ordinary skill in the art.

An “antibody” or “antibody molecule” is any immunoglobulin, including antibodies and fragments thereof, that binds to a specific antigen. The term includes polyclonal, monoclonal, chimeric, single domain (Dab) and bispecific antibodies. As used herein, antibody or antibody molecule contemplates recombinantly generated intact immunoglobulin molecules and immunologically active portions of an immunoglobulin molecule such as, without limitation: Fab, Fab′, F(ab′)₂, F(v), scFv, scFv₂, scFv-Fc, minibody, diabody, tetrabody, single variable domain (e.g., variable heavy domain, variable light domain), bispecific, Affibody® molecules (Affibody, Bromma, Sweden), and peptabodies (Terskikh et al. (1997) PNAS 94:1663-1668).

Chemotherapeutic agents are compounds that exhibit anticancer activity and/or are detrimental to a cell (e.g., a toxin). Suitable chemotherapeutic agents for use in the methods disclosed herein include, but are not limited to: toxins (e.g., saporin, ricin, abrin, ethidium bromide, diptheria toxin, Pseudomonas exotoxin, and others listed above); alkylating agents (e.g., nitrogen mustards such as chlorambucil, cyclophosphamide, isofamide, mechlorethamine, melphalan, and uracil mustard; aziridines such as thiotepa; methanesulphonate esters such as busulfan; nitroso ureas such as carmustine, lomustine, and streptozocin; platinum complexes such as cisplatin and carboplatin; bioreductive alkylators such as mitomycin, procarbazine, dacarbazine and altretamine); DNA strand-breakage agents (e.g., bleomycin); topoisomerase II inhibitors (e.g., amsacrine, dactinomycin, daunorubicin, idarubicin, mitoxantrone, doxorubicin, etoposide, and teniposide); DNA minor groove binding agents (e.g., plicamydin); antimetabolites (e.g., folate antagonists such as methotrexate and trimetrexate; pyrimidine antagonists such as fluorouracil, fluorodeoxyuridine, CB3717, azacitidine, cytarabine, and floxuridine; purine antagonists such as mercaptopurine, 6-thioguanine, fludarabine, pentostatin; asparginase; and ribonucleotide reductase inhibitors such as hydroxyurea); tubulin interactive agents (e.g., vincristine, vinblastine, and paclitaxel (Taxol)); hormonal agents (e.g., estrogens; conjugated estrogens; ethinyl estradiol; diethylstilbesterol; chlortrianisen; idenestrol; progestins such as hydroxyprogesterone caproate, medroxyprogesterone, and megestrol; and androgens such as testosterone, testosterone propionate, fluoxymesterone, and methyltestosterone); adrenal corticosteroids (e.g., prednisone, dexamethasone, methylprednisolone, and prednisolone); leutinizing hormone releasing agents or gonadotropin-releasing hormone antagonists (e.g., leuprolide acetate and goserelin acetate); and antihormonal antigens (e.g., tamoxifen, antiandrogen agents such as flutamide; and antiadrenal agents such as mitotane and aminoglutethimide). In a particular embodiment, the chemotherapeutic agent is selected from the group consisting of placitaxel (Taxon®), cisplatin, docetaxol, carboplatin, vincristine, vinblastine, methotrexate, cyclophosphamide, CPT-11, 5-fluorouracil (5-FU), gemcitabine, estramustine, carmustine, adriamycin (doxorubicin), etoposide, arsenic trioxide, irinotecan, and epothilone derivatives.

### II. USES OF Rpl22-ENCODING NUCLEIC ACIDS, Rpl22 PROTEINS AND ANTIBODIES THERETO

The identification of the role Rpl22 plays in T cell differentiation and transformation, provides utility for diagnosis, prognosis and gene therapy of cancer, including T-ALL and neuroblastoma. Isolation of Rpl22-encoding nucleic acids, proteins and antibodies thereto will also provide wide utility as prognostic indicators of neoplastic disease and as well as facilitate the development of therapeutic agents for the treatment of many types of cancer.

Additionally, Rpl22-related nucleic acids, proteins, and antibodies thereto, in accordance with this invention, may be used as research tools to identify other genes which encode proteins involved in the T-cell maturation.

### A. Rpl22-Encoding Nucleic Acids

Rpl22-encoding nucleic acids may be used for a variety of purposes in accordance with the present invention. Rpl22-encoding DNA, RNA, or fragments thereof may be used as probes to detect the presence of and/or expression of genes encoding the Rpl22 protein. Methods in which Rpl22-encoding nucleic acids may be utilized as probes for such assays include, but are not limited to: (1) in situ-hybridization; (2) Southern hybridization (3) Northern hybridization; and (4) assorted amplification reactions such as polymerase chain reactions (PCR).

The Rpl22-encoding nucleic acids of the invention may also be utilized as probes to identify related genes from other species as demonstrated herein. As is well known in the art, hybridization stringencies may be adjusted to allow hybridization of nucleic acid probes with complementary sequences of varying degrees of homology. Thus, Rpl22-encoding nucleic acids may be used to advantage to identify and characterize other genes of varying degrees of relation to Rpl22, thereby enabling further characterization of the observed altered gene expression involved in the aggressive progression of T-ALL for example. Additionally, they may be used to identify genes encoding proteins that interact with Rpl22 (e.g., by the “interaction trap” technique; see U.S. Pat. No. 5,580,736), which should further accelerate elucidation of these cellular signaling mechanisms which are involved in cancer progression (Golemis et al. 1996).

Nucleic acid molecules, or fragments thereof, encoding Rpl22 may also be utilized to control the production of Rpl22, thereby regulating the amount of protein available to participate in disease signaling pathways. Alterations in the physiological amount of Rpl22 protein may act synergistically with other agents used to halt tumor progression. In disease models of T-ALL, the nucleic acid molecules of the invention may be used to increase or decrease expression of Rpl22. In this embodiment, antisense molecules ShRNA or siRNA are employed which are targeted to expression-controlling sequences of Rpl22-encoding genes. Antisense, ShRNA or SiRNA oligonucleotides may be designed to hybridize to the complementary sequence of nucleic acid, pre-mRNA or mature mRNA, interfering with the production of polypeptide encoded by a given DNA sequence (e.g. either native Rpl22 polypeptide or a mutant or variant form thereof), so that its expression is reduced or prevented altogether. In addition to the Rpl22 coding sequence, antisense techniques can be used to target the control sequences of the Rpl22 gene, e.g. the 5′ flanking sequence of the Rpl22 coding sequence such as the translation start site. Antisense or SiRNA oligomers should be of sufficient length to hybridize to the target nucleotide sequence and exert the desired effect, e.g. blocking translation of a mRNA molecule. However, it should be noted that smaller oligomers are likely to be more efficiently taken up by cells in vivo such that a greater number of antisense oligomers may be delivered to the location of the target mRNA. Preferably, antisense oligomers should be at least 15 nucleotides long to achieve adequate specificity. Oligonucleotides for use in antisense technology are preferably between 15 to 30 nucleotides in length. The use of antisense molecules to decrease expression levels of a pre-determined gene is known in the art. The construction of antisense sequences and their use is described in Peyman and Ulman, Chemical Reviews, 90:543-584, (1990), Crooke, Ann. Rev. Pharmacol. Toxical., 32:329-376, (1992), and Zamecnik and Stephenson, P.N.A.S., 75:280-284, (1974). Antisense constructs may be generated which contain the entire Rpl22 cDNA in reverse orientation. Decreasing expression of Rpl22 in this controlled fashion will facilitate the identification of agents that can substitute for Rpl22 protein function, thereby providing new agents for the treatment of proliferative disorders, including cancer.

In another embodiment, overexpression of the Rpl22 gene will be introduced into T cells in experiments to assess restoration of Rpl22 activity in such cells as overexpression can lead to overproduction of the encoded protein, Rpl22. Overproduction of Rpl22 in cells may be assessed by immunofluorescence or any other standard technique known in the art. Alternatively, overexpression of Rpl22 by this method may facilitate the isolation and characterization of other components involved in the protein-protein complex formation that occurs as a cell progressively becomes more malignant.

As described above, Rpl22-encoding nucleic acids are also used to advantage to produce large quantities of substantially pure Rpl22 protein, or selected portions thereof.

### B. Rpl22 Protein and Antibodies

Purified Rpl22 protein, or fragments thereof, may be used to produce polyclonal or monoclonal antibodies which also may serve as sensitive detection reagents for the presence and accumulation of Rpl22 (or complexes containing Rpl22) in biopsy samples or cultured cells. Recombinant techniques enable expression of fusion proteins containing part or all of the Rpl22 protein. The full length protein or fragments of the protein may be used to advantage to generate an array of monoclonal antibodies specific for various epitopes of the protein, thereby providing even greater sensitivity for detection of the protein in T cells.

Polyclonal or monoclonal antibodies immunologically specific for Rpl22 may be used in a variety of assays designed to detect and quantitate the protein. Such assays include, but are not limited to: (1) flow cytometric analysis; (2) immunochemical localization of Rpl22 in cells; and (3) immunoblot analysis (e.g., dot blot, Western blot) of extracts from cells. Additionally, as described above, anti-Rpl22 can be used for purification of Rpl22 (e.g., affinity column purification, immunoprecipitation).

From the foregoing discussion, it can be seen that Rpl22-encoding nucleic acids, Rpl22 expressing vectors, Rpl22 proteins and anti-Rpl22 antibodies of the invention can be used to detect Rpl22 gene expression and alter Rpl22 protein accumulation for purposes of assessing those patients at risk for T-ALL progression and relapse. Indeed, we have produced an anti-Rpl22 polyclonal antibody in rabbits useful for immunoprecipitating Rpl22 from biological samples. The invention also provides materials that facilitate the elucidation of the genetic and protein interactions involved in the regulation of the disease progression as a normal T cell gives rise to a malignancy.

Exemplary approaches for detecting Rpl22-encoding nucleic acid molecules or polypeptides/proteins include:

a) determining the presence, in a sample from a patient, of nucleic acid molecules according to the present invention; or
b) determining the presence, in a sample from a patient, of the polypeptide encoded by the Rpl22 gene and, if present, determining whether the polypeptide is full length, and/or is mutated, and/or is expressed at the normal level; or
c) using DNA restriction mapping to compare the restriction pattern produced when a restriction enzyme cuts a sample of nucleic acid molecules from the patient with the restriction pattern obtained from the Rpl22-encoding nucleic acid sequence; or,
d) using a specific binding member capable of binding to a Rpl22 nucleic acid sequence, the specific binding member comprising nucleic acid hybridizable with the Rpl22 sequence, or substances comprising an antibody domain with specificity for a Rpl22 nucleic acid sequence or the polypeptide encoded by it, the specific binding member being labeled so that binding of the specific binding member to its binding partner is detectable; or,
e) using PCR involving one or more primers based on Rpl22 nucleic acid sequences to screen for Rpl22 sequences in a sample from a patient.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples and they do not need to be listed here. Further, the term “specific binding pair” is also applicable where either or both of the specific binding member and the binding partner comprise a part of a large molecule. In embodiments in which the specific binding pair are nucleic acid sequences, they will be of a length to hybridize to each other under conditions of the assay, preferably greater than 10 nucleotides long, more preferably greater than 15 or 20 nucleotides long.

In most embodiments for screening for cancer susceptibility alleles, the Rpl22-encoding nucleic acid molecules in the sample will initially be amplified, e.g. using PCR, to increase the amount of the analyte as compared to other sequences present in the sample. This allows the target sequences to be detected with a high degree of sensitivity if they are present in the sample. This initial step may be avoided by using highly sensitive array techniques that are becoming increasingly important in the art.

The identification of the Rpl22-encoding nucleic acid sequence and its association with T-ALL paves the way for aspects of the present invention to provide the use of materials and methods, such as are disclosed and discussed above, for establishing the presence or absence in a test sample of the Rpl22-encoding nucleic acid molecule, in particular an allele or variant specifically associated with cancer, especially T-ALL. This may be for diagnosing a predisposition of an individual to cancer. It may be for diagnosing cancer of a patient with the disease as being associated with Rpl22 or for diagnosing a propensity for relapse of T-ALL.

This allows for planning of appropriate therapeutic and/or prophylactic measures, permitting stream-lining of treatment. The approach can also stream-line treatment by targeting those patients most likely to benefit.

According to another aspect of the invention, methods of screening drugs for cancer therapy to identify suitable drugs for restoring Rpl22 product function are provided. Restoration of Rpl22 function by gene transfer or by pharmacological means (e.g., small molecules which mimic Rpl22 structure and/or function) would be expected to ameliorate the aberrant growth characteristics of T-ALL cells.

The Rpl22 polypeptide or fragment employed in drug screening assays may either be free in solution, affixed to a solid support or within a cell. One method of drug screening utilizes eukaryotic or prokaryotic host cells which are stably transformed with recombinant polynucleotides expressing the polypeptide or fragment, preferably in competitive binding assays. Such cells, either in viable or fixed form, can be used for standard binding assays. One may determine, for example, formation of complexes between a Rpl22 polypeptide or fragment and the agent being tested, or examine the degree to which the formation of a complex between an Rpl22 polypeptide (wild-type or mutated) or fragment and a known ligand or binding partner (e.g., p53) is interfered with by the agent being tested.

Another technique for drug screening provides high throughput screening for compounds having suitable binding affinity to the Rpl22 polypeptides and is described in detail in Geysen, PCT published application WO 84/03564, published on Sep. 13, 1984. Briefly stated, large numbers of different, small peptide test compounds are synthesized on a solid substrate, such as plastic pins or some other surface. The peptide test compounds are reacted with Rpl22 polypeptide and washed. Bound Rpl22 polypeptide is then detected by methods well known in the art.

Purified Rpl22 can be coated directly onto plates for use in the aforementioned drug screening techniques. However, non-neutralizing antibodies to the polypeptide can be used to capture antibodies to immobilize the Rpl22 polypeptide on the solid phase.

Molecular modeling should facilitate the identification of specific organic molecules with capacity to bind to the active site of the altered Rpl22 proteins based on conformation or key amino acid residues required for function. A combinatorial chemistry approach will be used to identify molecules with greatest activity and then iterations of these molecules will be developed for further cycles of screening. In certain embodiments, candidate drugs can be screened from large libraries of synthetic or natural compounds. One example is an FDA approved library of compounds that can be used by humans. In addition, compound libraries are commercially available from a number of companies including but not limited to Maybridge Chemical Co. (Trevillet, Cornwall, UK), Comgenex (Princeton, N.J.), Microsource (New Milford, Conn.), Aldrich (Milwaukee, Wis.), AKos Consulting and Solutions GmbH (Basel, Switzerland), Ambinter (Paris, France), Asinex (Moscow, Russia), Aurora (Graz, Austria), BioFocus DPI, Switzerland, Bionet (Camelford, UK), ChemBridge, (San Diego, Calif.), ChemDiv, (San Diego, Calif.), Chemical Block Lt, (Moscow, Russia), ChemStar (Moscow, Russia), Exclusive Chemistry, Ltd (Obninsk, Russia), Enamine (Kiev, Ukraine), Evotec (Hamburg, Germany), Indofine (Hillsborough, N.J.), Interbioscreen (Moscow, Russia), Interchim (Montlucon, France), Life Chemicals, Inc. (Orange, Conn.), Microchemistry Ltd. (Moscow, Russia), Otava, (Toronto, ON), PharmEx Ltd. (Moscow, Russia), Princeton Biomolecular (Monmouth Junction, N.J.), Scientific Exchange (Center Ossipee, N.H.), Specs (Delft, Netherlands), TimTec (Newark, Del.), Toronto Research Corp. (North York ON), UkrOrgSynthesis (Kiev, Ukraine), Vitas-M, (Moscow, Russia), Zelinsky Institute, (Moscow, Russia), and Bicoll (Shanghai, China).

Libraries of natural compounds in the form of bacterial, fungal, plant and animal extracts are commercially available or can be readily prepared by methods well known in the art. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Several commercial libraries can be used in the screens.

A further technique for drug screening involves the use of host eukaryotic cell lines or cells (such as described above) which have a nonfunctional Rpl22 gene. These host cell lines or cells are defective at the Rpl22 polypeptide level. The host cell lines or cells are grown in the presence of drug compound. The rate of growth of the host cells is measured to determine if the compound is capable of regulating the growth of Rpl22 defective cells.

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. See, e.g., Hodgson, (1991) Bio/Technology 9: 19-21. In one approach, one first determines the three-dimensional structure of a protein of interest (e.g., Rpl22 polypeptide) or, for example, of the Rpl22-DNA complex, by x-ray crystallography, by nuclear magnetic resonance, by computer modeling or most typically, by a combination of approaches. Less often, useful information regarding the structure of a polypeptide may be gained by modeling based on the structure of homologous proteins. An example of rational drug design is the development of HIV protease inhibitors (Erickson et al., (1990) Science 249:527-533). In addition, peptides (e.g., Rpl22 polypeptide) may be analyzed by an alanine scan (Wells, 1991) Meth. Enzym. 202:390-411. In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original molecule. The anti-id could then be used to identify and isolate peptides from banks of chemically or biologically produced banks of peptides. Selected peptides would then act as the pharmacore.

Thus, one may design drugs which have, e.g., improved Rpl22 polypeptide activity or stability or which act as inhibitors, agonists, antagonists, etc. of Rpl22 polypeptide activity. By virtue of the availability of cloned Rpl22 sequences, sufficient amounts of the Rpl22 polypeptide may be made available to perform such analytical studies as x-ray crystallography. In addition, the knowledge of the Rpl22 protein sequence provided herein will guide those employing computer modeling techniques in place of, or in addition to x-ray crystallography.

The present invention further provides “compositions” in biological compatible solution, pharmaceutically acceptable excipient, carrier, buffer, stabilizer or other materials well known to those skilled in the art, comprising the nucleic acids, polypeptides, vectors or antibodies of the invention. A biologically compatible solution is a solution in which the polypeptide, nucleic acid, vector, or antibody of the invention is maintained in an active form, e.g. in a form able to effect a biological activity. Generally, such a biologically compatible solution will be an aqueous buffer, e.g. Tris, phosphate, or HEPES buffer, containing salt ions. Usually the concentration of salt ions will be similar to physiological levels. Biologically compatible solutions may include stabilizing agents and preservatives.

Such compositions may be formulated for administration by topical, oral, parenteral, intranasal, subcutaneous, and intraocular routes. Parenteral administration is meant to include intravenous injection, intramuscular injection, intraarterial injection or infusion techniques. The compositions may be administered parenterally in dosage unit formulations containing standard well known non-toxic physiologically acceptable carriers, adjuvants and vehicles as desired.

The preferred sterile injectable preparations may be a solution or suspension in a nontoxic parenterally acceptable solvent or diluent. Examples of pharmaceutically acceptable carriers are saline, buffered saline, isotonic saline (e.g. monosodium or disodium phosphate, sodium, potassium, calcium or magnesium chloride, or a mixture or such salts), Ringers solution, dextrose, water, sterile water, glycol, ethanol, and combinations thereof. 1,3-butanediol and sterile fixed oils are conveniently employed as solvents or suspending media. Any bland fixed oil may be employed including synthetic mono- or di-glycerides. Fatty acids such as oleic acid also find use in the preparation of injectables.

The present invention provides “methods of treatment” which comprise the administration to a human or other animal of an effective amount of a composition of the invention.

Effective amounts vary, depending on the age, type and severity of the condition to be treated, body weight, desired duration of treatment, method of administration, and other parameters. Effective amounts are determined by a physician or other qualified medical professional.

The Rpl22 polypeptides of the invention may also be administered via injection in a biologically compatible buffer, in doses of about 0.01 mg/kg to about 100 mg/kg, preferably about 0.1 mg/kg to about 50 mg/kg, and most preferably about 1 mg/kg to about 10 mg/kg of body weight per day. Alternatively, nucleic acids expressing the peptides of the invention may be delivered directly to a patient in vectors or liposomes which facilitate entry into a cancer cell.

The following example is provided to facilitate the practice of the present invention. It is not intended to limit the invention in any way.

### Example I

### Rpl22 is a Biomarker for Cancer, Particularly T-ALL

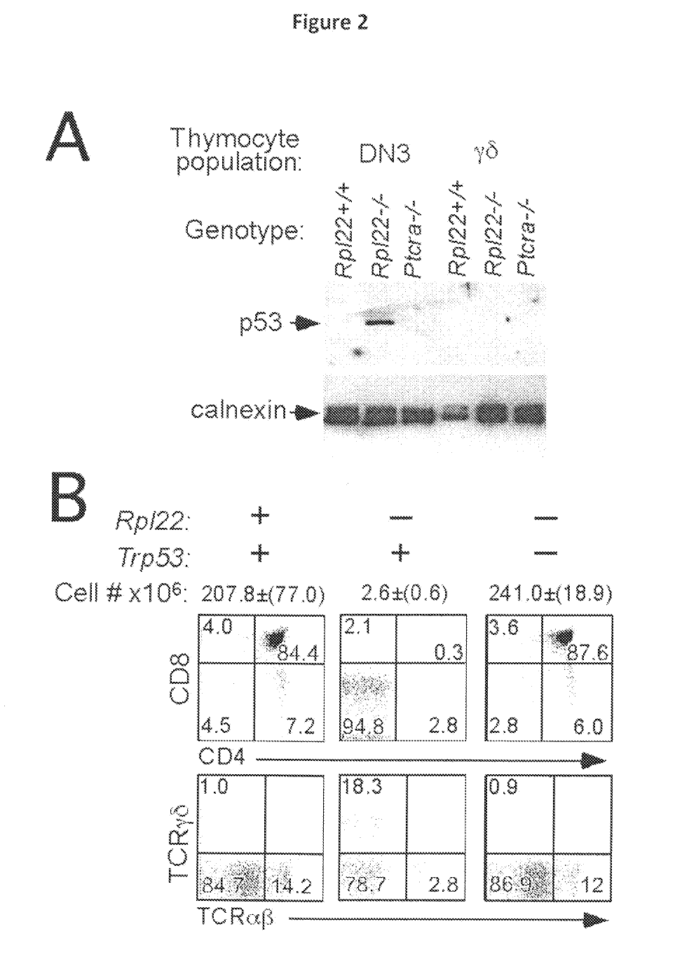

Rpl22 is a component of the 60S large ribosomal subunit and is ubiquitously expressed, but is not required for CAP-dependent translation (J. P. Lavergne, F. Conquet, J. P. Reboud et al., FEBS Lett 216 (1), 83 (1987); however, Rpl22 can stimulate IRES-dependent translation (J. Wood, R. M. Frederickson, S. Fields et al., J Virol 75 (3), 1348 (2001). While Rpl22 has been suggested to play a non-essential role in ribosome assembly or function or in the assembly of other multi-subunit ribonucleoprotein (RNP) particles within the cell (S. Le, R. Sternglanz, and C. W. Greider, Mol Biol Cell 11 (3), 999 (2000); C. Shu-Nu, C. H. Lin, and A. Lin, FEBS Lett 484 (1), 22 (2000), Rpl22 had not been linked to any specific developmental pathway, mechanism, or disease. We have now shown that mice in which the RPL22 gene has been disrupted by gene-trap mutagenesis are viable, fertile, and grossly normal. This is the only example to date in which ablation of a mammalian ribosomal protein gene was not lethal. In fact the only obvious defect that we have been able to attribute to Rpl22-deficiency to date is a very specific block in development of αβ lineage T cells. The blockade in development of αβ T cells is caused by selective activation of a p53 dependent checkpoint. Indeed, p53 induction is not observed in γδ lineage cells which are not arrested, but is observed in the particular DN subset where arrest occurs (FIG. 2A). Interestingly, p53 induction in Rpl22-deficient thymocytes appears to be associated with increased p53 synthesis, not decreased turnover. Development of Rpl22-deficient thymocytes is completely rescued by p53-deficiency, indicating that p53 is an important target of Rpl22 (FIG. 2B). Taken together, these data suggest that Rpl22 function is important for αβ T development, but is dispensable in most other cell types.

Loss of One Allele of RPL22 Accelerates Development of Thymic Lymphoma in Myr-Akt2 Transgenic Mice.

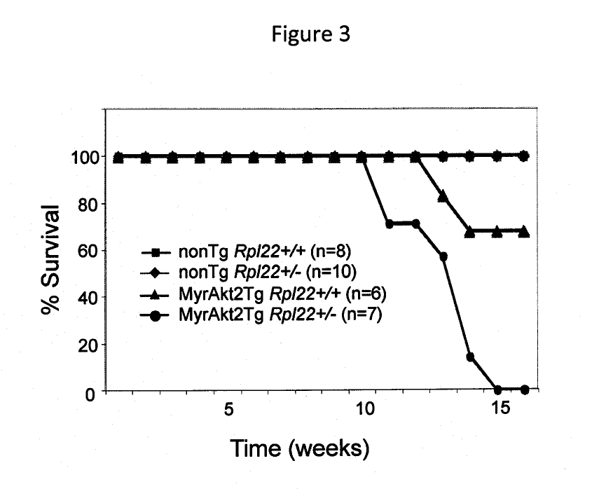

Because ribosomal proteins have been implicated as haploinsufficient tumor suppressors in zebrafish (A. Amsterdam, K. C. Sadler, K. Lai et al., PLoS Biol 2 (5), E139 (2004), we asked whether inactivation of one Rpl22 allele might influence the development of thymic lymphoma in mice. Development of thymic lymphoma was chosen for analysis because Rpl22-deficiency selectively blocks T cell development. In doing so, we employed Tg mice in which expression of activated Akt2 (membrane-targeted by myristoylation; Myr-Akt2) is enforced in immature T cell progenitors because: 1) the Akt signaling pathway is frequently activated in T-ALL; 2) Myr-Akt2 is highly oncogenic and causes thymic lymphomas in 80% of mice by 17-18 weeks of age; 3) the resultant thymic lymphomas are invariably associated with a translocation that results in induction in myc expression. Upon crossing the Myr-Akt2 Tg with Rpl22+/− mice, we generated a cohort of offspring which were monitored for development of disease (FIG. 3). It is clear that loss of one of the Rpl22 alleles is sufficient to accelerate development of thymic lymphoma in this model.

### Rpl22 Mutations in T-ALL.

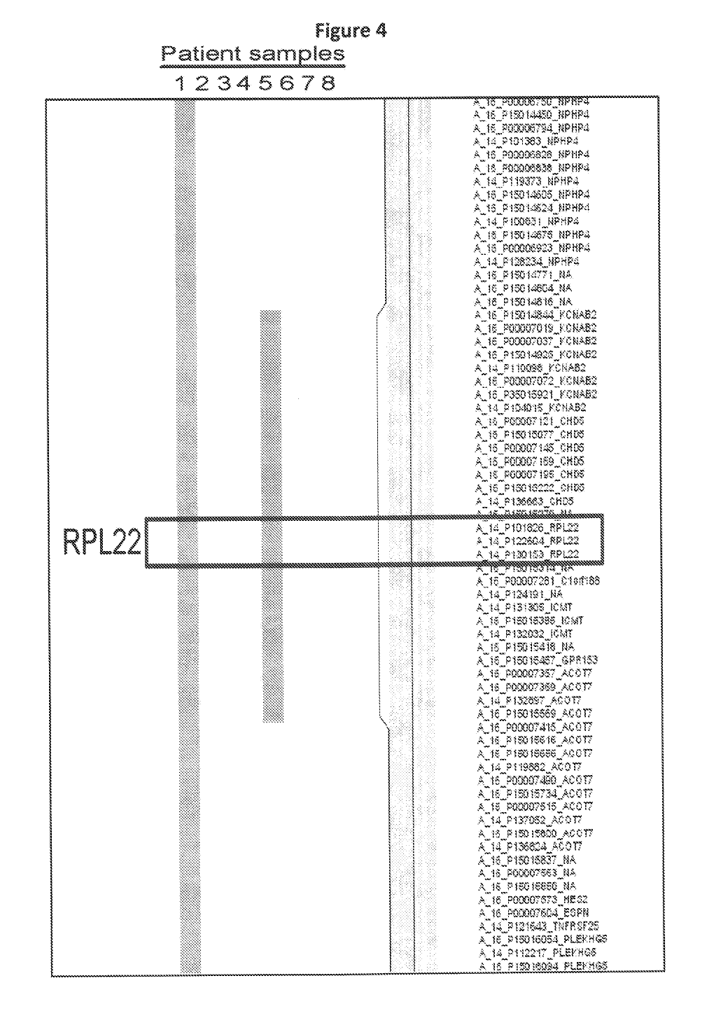

Our data indicate that loss of one Rpl22 allele predisposes mice to develop thymic lymphoma induced by active Akt. Since mutations that activate Akt (e.g., Notch activation or PTEN loss) are common in T-ALL, we asked whether Rpl22 might also be inactivated in primary T-ALL samples. To address this issue, aCGH analysis was performed using Agilent arrays as described on a small cohort (8) of primary T-ALL patient samples to determine if the Rpl22 locus (1p36.3-p36.2) was affected (FIG. 4). Interestingly, 2 of the 8 samples analyzed exhibited deletion of one Rpl22 allele (FIG. 4; dark blue bars). One patient (#5) exhibited a focal deletion which eliminated Rpl22 along with 4 or 5 other genes, while the deletion found in the other patient (#1) was much larger.

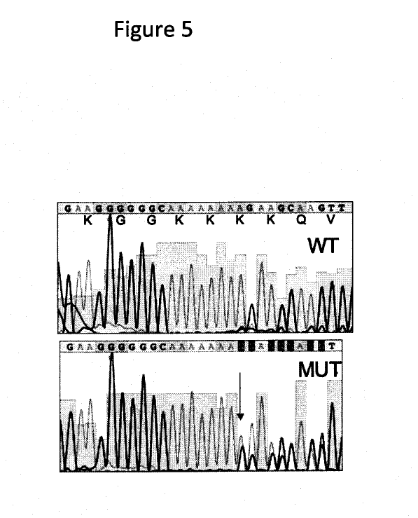

An extension of this study involved performance of ACGH on 48 primary T-ALL patient samples. Five of these were found to have deletions on one Rpl22 allele. Two patients exhibited focal deletions which eliminated Rpl22 along with 4 or 5 other genes, while the deletions found in the other 3 patients were much larger. Interestingly, 4 of the 5 deletions occurred in patients that succumbed to the disease (i.e., IF, induction failure; or REL, relapse). The association of loss of Rpl22 with poor prognosis already approaches statistical significance (p<0.07) despite the relatively small sample population. Therefore, loss of one Rpl22 allele is associated with adverse disease outcome. To determine if there were specific mutations in the Rpl22 gene in particular, we also performed sequencing analysis of the Rpl22 gene in a panel of 19 T-ALL cell lines independently derived from relapsed patients. Interestingly, in ⅙ of the cell lines, we found a single nucleotide deletion in a run of As that caused a frameshift in Rpl22, resulting in premature termination after amino acid 18 (FIG. 5, “V” in top panel). The deletion occurred on only one allele resulting in a mixed sequence past the point of deletion, as indicated by the Ns and double peaks (FIG. 5, bottom panel). The presence of deletions encompassing the Rpl22 locus in the most intractable cases of T-ALL coupled with the high frequency (˜15%) of inactivating point mutations in T-ALL lines derived from relapse patients suggests that genetic events inactivating the Rpl22 gene provide a biomarker for aggressive disease.

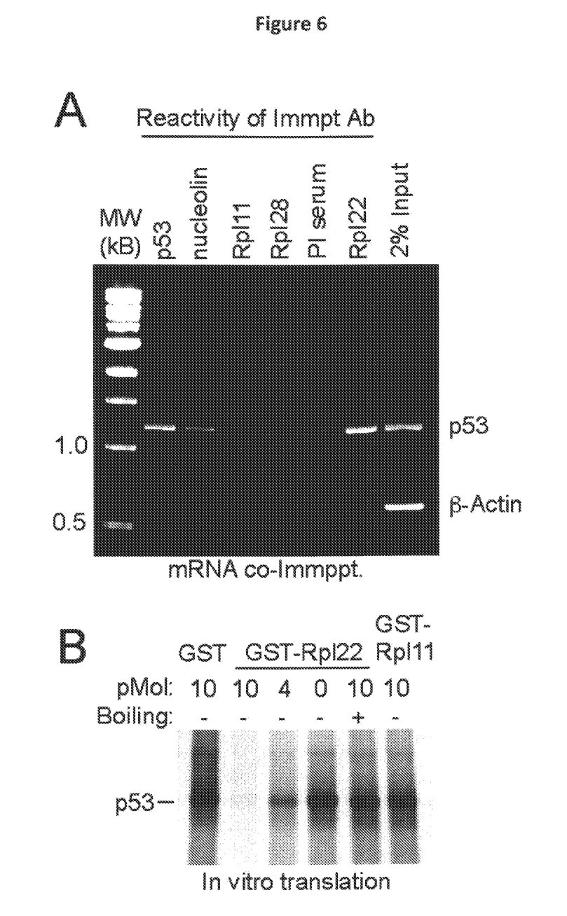

As mentioned above, it appears that Rpl22 is involved in regulation of expression of p53. While it is becoming more widely accepted that mutations in ribosomal proteins play a role in the etiology of some cancers, it remains unclear how they do so. We hypothesize that a likely explanation is that particular ribosomal proteins are involved in controlling the translation of key molecules regulating cell growth and survival. Accordingly, expression of those molecules is then perturbed under conditions where expression of that ribosomal protein is altered by mutation. Indeed, the data presented herein demonstrate that Rpl22 plays a role in regulating the expression of the tumor suppressor p53 in that Rpl22-deficiency causes an increase in p53 translation in developing T lymphocytes (thymocytes) See FIG. 2. To address whether Rpl22 regulated translation of p53 mRNA indirectly through a succession of intermediates or directly through binding to p53 mRNA, we performed co-immunoprecipitation analysis using an anti-Rpl22 antibody (Ab) which we raised against the N-terminal 15aa of Rpl22. Consistent with regulation through direct binding, anti-Rpl22 Ab co-precipitated p53 mRNA (but not β-actin mRNA) from cytosolic detergent extracts of developing thymocytes (FIG. 6A). By comparison to input mRNA, we deduced that 2-5% of cellular p53 mRNA was able to withstand stringent washes which disrupt the integrity of intact ribosomes (1M urea), thereby providing a minimum estimate of the fraction of the p53 mRNA pool stably associated with Rpl22 (FIG. 6A). Co-precipitation of p53 mRNA is blocked by pretreatment of anti-Rpl22 with the peptide immunogen (data not shown) and is a selective capability of Rpl22, as other ribosomal proteins were not found to associate (Rpl11, Rpl28, Rps6)(FIG. 6A and data not shown). Rpl22 is an RNA binding protein whose specificity was evaluated by Selex analysis, which suggested that Rpl22 recognizes a stem loop structure with a G-C at the neck followed by U (M. Dobbelstein and T. Shenk et al. J Virol 69 (12), 8027 (1995).

Using an RNA secondary structure prediction algorithm (P-fold), we identified 4 potential binding sites in p53 mRNA that resemble an Rpl22-binding motif, suggesting that Rpl22 might interact directly with p53 mRNA. To test whether Rpl22 could inhibit p53 translation we treated in vitro transcription/translation reactions of a p53 template with recombinant Rpl22. Indeed, exogenous GST-Rpl22 fusion protein (but not GST-Rpl1) suppressed the translation of p53 mRNA in vitro in a dose-dependent manner (FIG. 6B). The ability of recombinant Rpl22 to suppress the translation of p53 mRNA in vitro provides an explanation for the increased translation (translational de-repression) of p53 in Rpl22-deficient thymocytes (S. J. Anderson et al., Immunity 26 (6), 759 (2007)).

It should be noted that we have not found any evidence of increased p53 expression in Rpl22+/− thymocytes and so we do not think that the translational regulation of p53 is involved in Rpl22's proposed role as a haploinsufficent tumor suppressor in T cell lymphoma and T-ALL. Nevertheless, the ability of Rpl22 to translationally regulate p53 expression provides precedent for a mechanism by which ribosomal proteins regulate transformation, i.e. translational control of oncogenes or tumor suppressors.

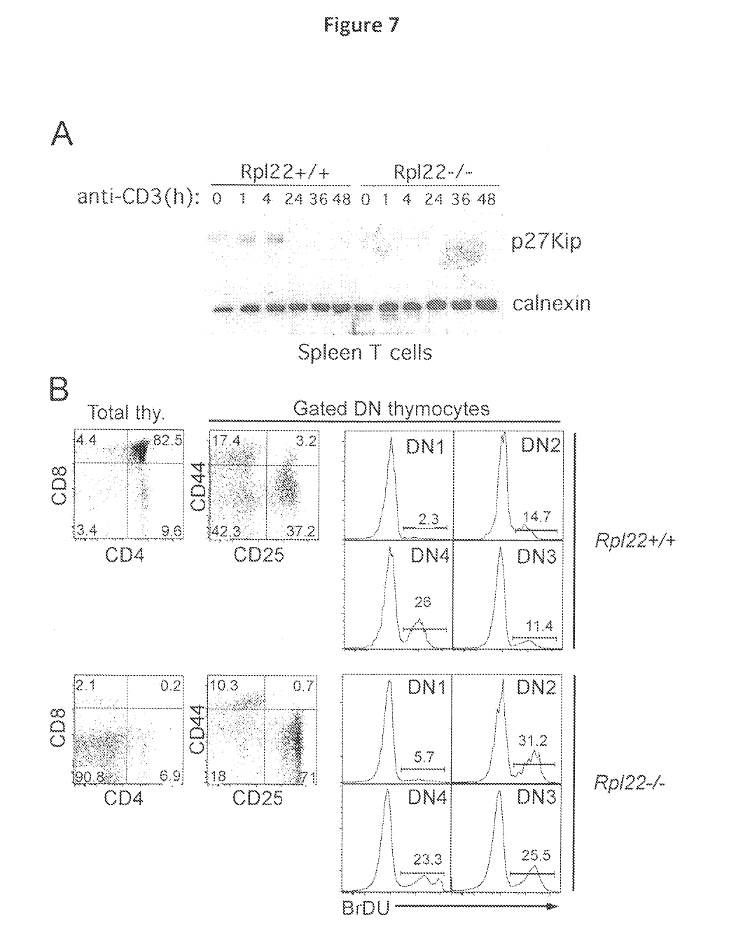

p27Kip expression is impaired in Rpl22-deficient mice. The cyclin-dependent kinase inhibitor p27Kip is a tumor suppressor whose loss is a negative prognostic indicator in human cancers; however, misregulation rather than gene loss is responsible for decreased p27 levels in most tumors (J. Tsihlias et al., Annu Rev Med 50, 401 (1999)). Interestingly, one way in which p27Kip expression is regulated is through CAP-independent, IRES-dependent translation (M. Kullmann et al., Genes Dev 16 (23), 3087 (2002)). Internal ribosome entry sites or IRES elements are thought to be involved in translational regulation of as much as 10% of the genome with many IRES-dependent proteins playing key regulatory roles in growth and survival: Bcl-X_{L}, Bcl-2, myb, RUNX1, BAG-1, Notch2, and Pim1. Rpl22 is not an essential gene in yeast and is not thought to be required for global or CAP-dependent translation. In agreement, we have found that global protein synthesis is not markedly altered by Rpl22-deficiency (FIG. 6.) Nevertheless, Rpl22 has been implicated in IRES-dependent translation as Rpl22 overexpression can stimulate IRES-dependent translation from the 3′UTR of Hepatitis C Virus. Because of the possible role of Rpl22 in regulating IRES-dependent translation, we asked whether p27Kip expression might be impaired by Rpl22-deficiency. p27Kip is expressed in spleen T cells but expression is extinguished during induction of proliferation by treatment with anti-CD3 Ab (FIG. 7A, left side of upper panel). Importantly, we found that expression of p27Kip protein was already markedly reduced in Rpl22−/− T cells even before stimulation (FIG. 7A, right side of upper panel). Because of the reduction in p27Kip levels in mature T cells, we asked if developing T cells from Rpl22−/− mice might exhibit signs of altered cell cycle status. As described previously, thymocyte development is arrested in Rpl22−/− mice, with cellularity decreased ˜100-fold and apoptosis increased substantially. Nevertheless, despite the striking arrest of T cell development and reduced cellularity in Rpl22−/− mice (most cells are CD4−CD8−), Rpl22−/− thymic subsets exhibited increased incorporation of Bromodeoxyuridine (BrDU) during the 4 h pulse (FIG. 7B). Both CD4−CD8− (double negative or DN) thymocytes that are CD44+ CD25+ (DN2) as well as those which are CD44−CD25+ (DN3) from Rpl22−/− mice had a greater proportion of BrdU+ cells than did their Rpl22+/+ littermates, suggesting that more cells were in S-phase at the time of analysis. Taken together, these data suggest that the increased mitotic index of DN2 and DN3 cells from Rpl22−/− mice might result from impaired expression of p27Kip. Thus, it appears that Rpl22 regulates the translation of a selected subset of mRNAs involved in controlling thymocyte growth, survival, and transformation. Further characterization of this set of genes that are controlled by Rpl22 should facilitate establishment of an expression signature that will lend insight into the etiology of lymphomagenesis as well as having prognostic value regarding disease progression or drug responsiveness in T-ALL.

### Rpl22 Functions in Transformation

We also performed experiments to assess whether Rpl22 mutations predisposed Rpl22 null MEF cells to cancer using conventional cellular assays. Rpl22+/− male and female mice were mated for 12 h and then separated in order to limit the time interval during which pregnancy occurred. Pregnant females were sacrificed at gestation day 13.5. Fetal mice were surgically removed, following which the genotype of the mouse fetuses (E13.5-14) was determined and the head and major internal organs removed (Anderson, Immunity 26 (6), 759 (2007). Mouse embryonic fibroblasts (MEF) were proteolytically isolated from the trunks of the fetuses by digestion with 0.4% trypsin for 15 min at 37° C. MEF from Rpl22+/+, Rpl22+/−, and Rpl22−/− fetuses were immortalized by serial passage (more than 9 times) in Iscove's Modified media containing 10% Fetal Bovine Serum and standard additives.

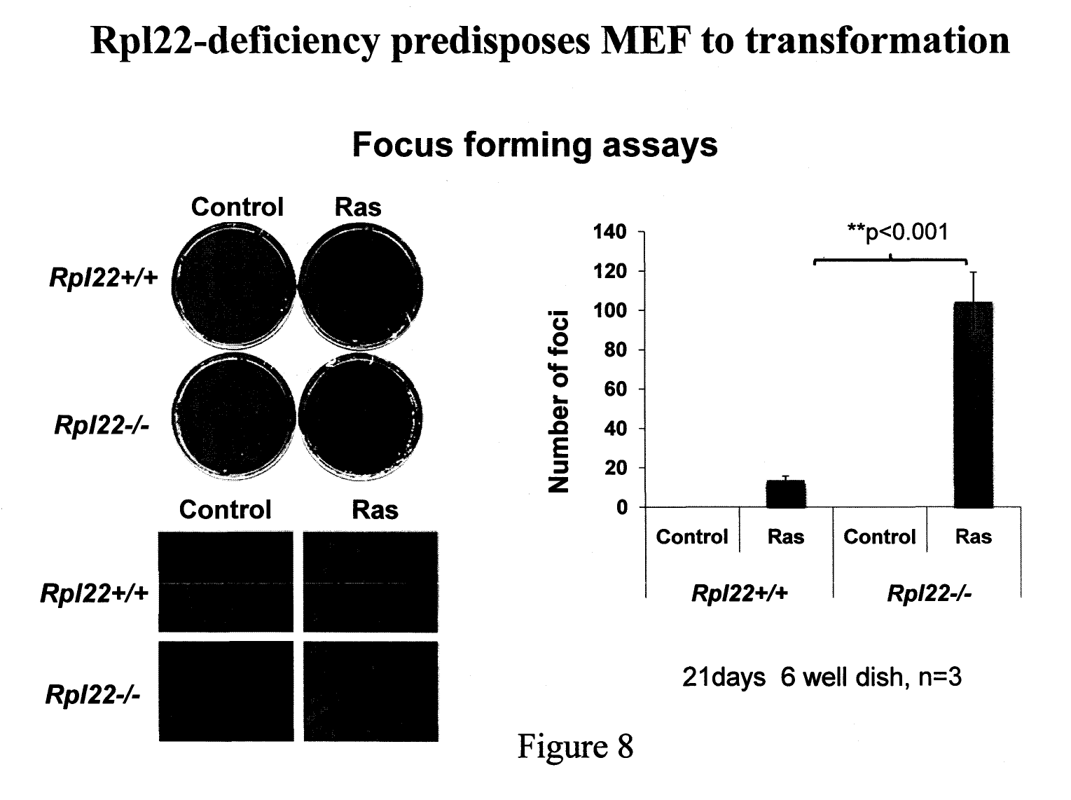

Using these MEFs, we found that Rpl22 inactivation promotes transformation in acute assays of transformation in vitro. Immortalized Rpl22+/+ and Rpl22−/− MEF cells were infected with control retroviral vectors encoging oncogenic Ras (H-Ras G12V) or control(puro) (Zhang, Dev. Cell 8 (1), 19 (2005) and selected with puromycin for about a week. Then cells maintained in puromycin supplemented medium were counted and seeded into 6-well-plates in triplicate. Culture medium was changed every 2-3 days until the assays were harvested, 2-3 weeks later. After washing with PBS, colonies were stained with 0.5% crystal violate, following which foci were counted. See FIG. 8. Similar results were obtained using a soft agar colony assay in which cells transduced as above, mixed with an equal volume of 0.7% noble agar, seeded at a density of 10000 cells in 35 mm Petri plates containing a base layer of 0.5% noble agar, and crystal violet stained colonies were enumerated after 30 d. The data show a significant enhancement of focus formation in ras transformed MEF cells lacking Rpl22.

Multiple members of the let-7 family of miRNAs are often repressed in human cancers, thereby promoting oncogenesis by derepressing targets such as HMGA2, K-Ras and c-Myc. However, the mechanism by which let-7 miRNAs are coordinately repressed is unclear. The RNA-binding proteins LIN28 and LIN28B block let-7 precursors from being processed to mature miRNAs, suggesting that their overexpression might promote malignancy through repression of let-7. Viswanathan et al. (Nat Genet. (2009) 41:843-8) have shown that LIN28 and LIN28B are overexpressed in primary human tumors and human cancer cell lines (overall frequency approximately 15%), and that overexpression is linked to repression of let-7 family miRNAs and derepression of let-7 targets. LIN28 and LIN28B facilitate cellular transformation in vitro, and overexpression is associated with advanced disease across multiple tumor types.

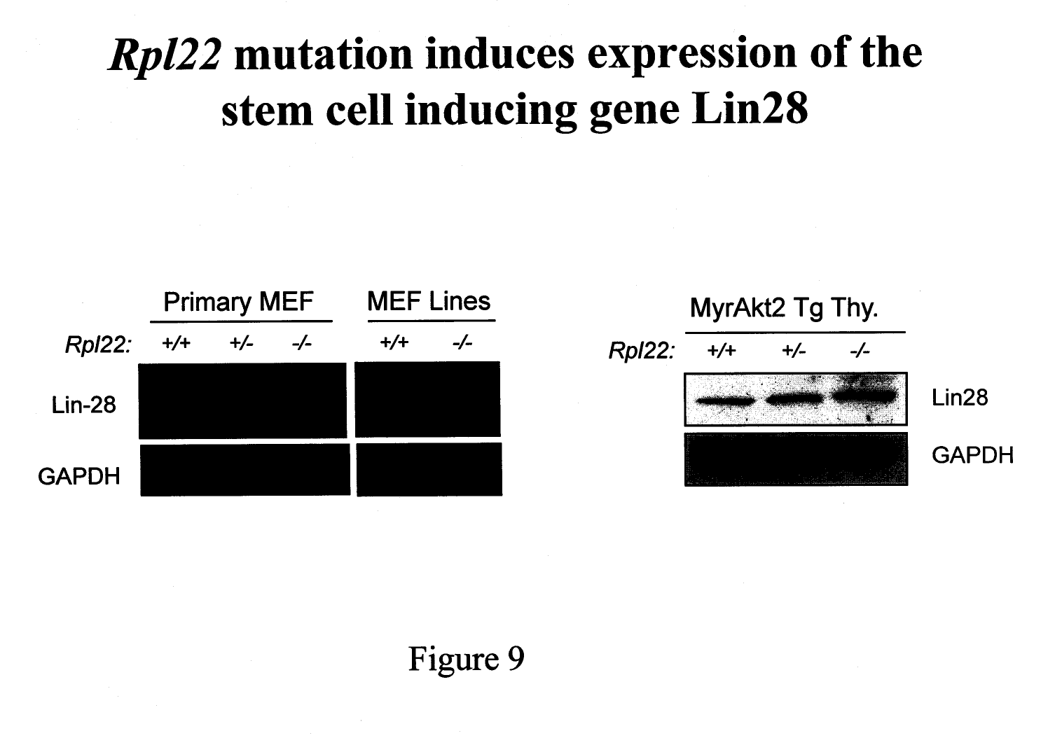

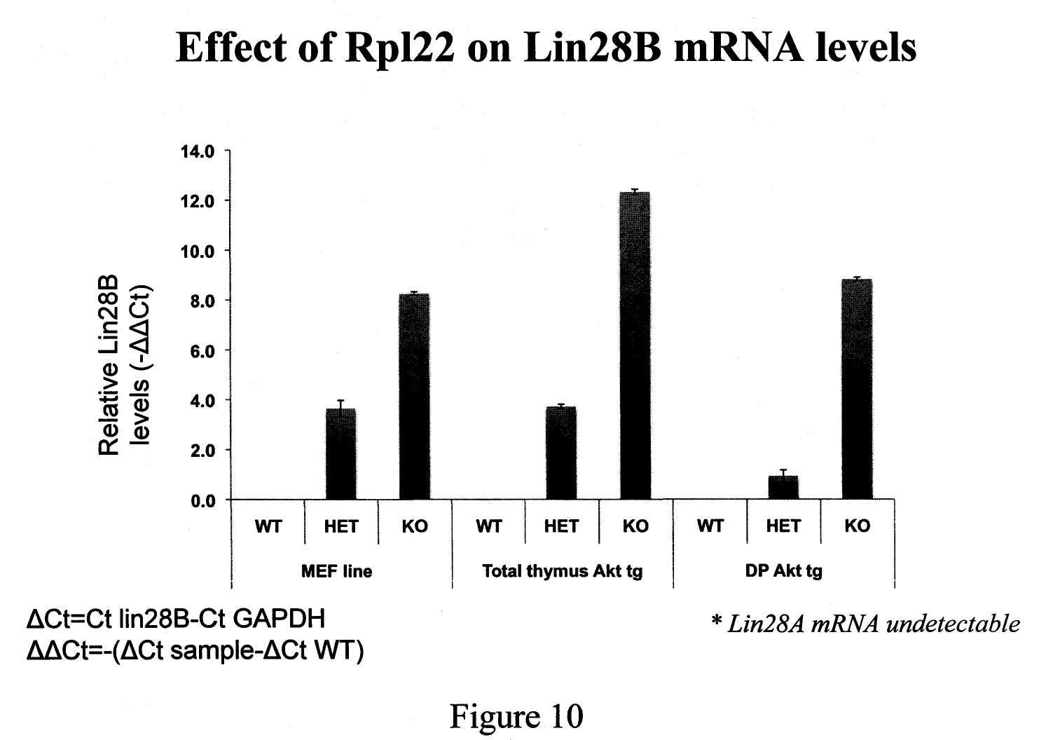

We have found that Rpl22 inactivation induces expression of Lin28B expression. As shown in FIG. 9, Rpl22 inactivation results in the induction of Lin28B in primary MEF, immortalized MEF lines, and primary thymocytes expressing oncogenic Akt (myrystoylated Akt2; MyrAkt2). Detergent extracts from Rpl22+/+, +/−, and −/− cells were immunoblotted with antibodies reactive with Lin28B as well as the GAPDH loading control. Bound antibody was visualized using fluorescently labeled secondary antibodies and the Odyssey imaging system. In parallel, mRNA levels encoding Lin28B were evaluated by real time PCR using real time PCR primers and probes commercially available from ABI. See FIG. 10.

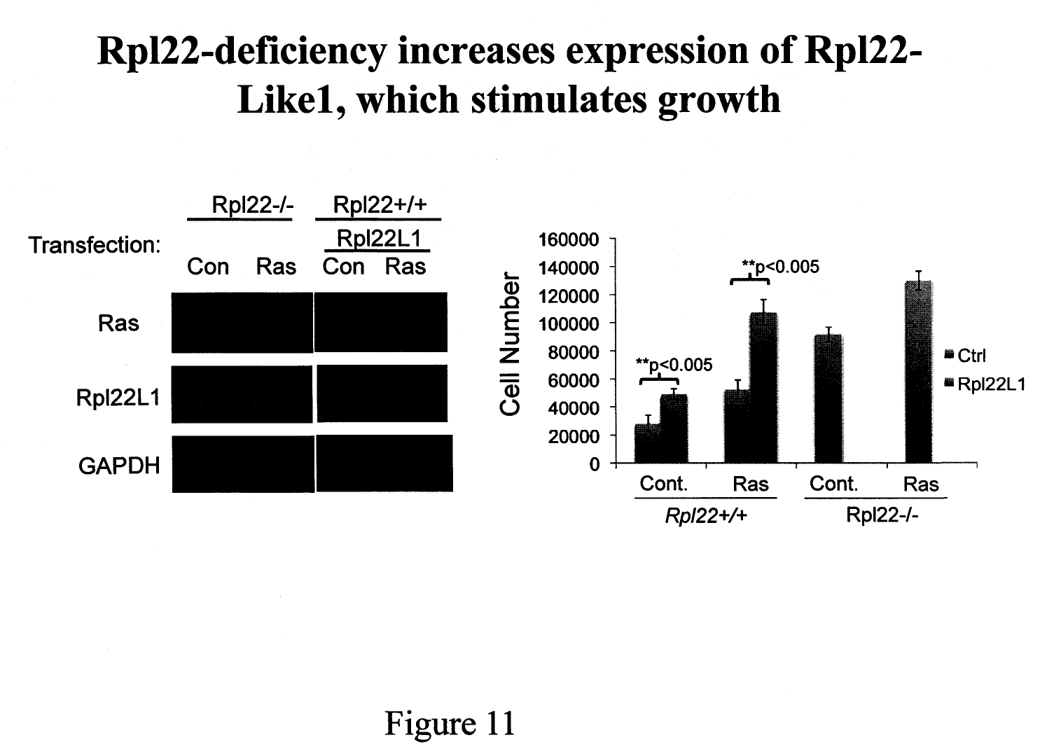

In additional studies, we have found that in Rpl22−/− MEF lines, expression of the paralog of Rpl22, Rpl22-Like 1, is dramatically increased. See FIG. 11. This increase in Rpl22L1 is associated with increased growth of Rpl22−/− MEF lines. Moreover, Rpl22-like1 overexpression is able to confer this accelerated growth property on Rpl22+/+ MEF upon ectopic expression.

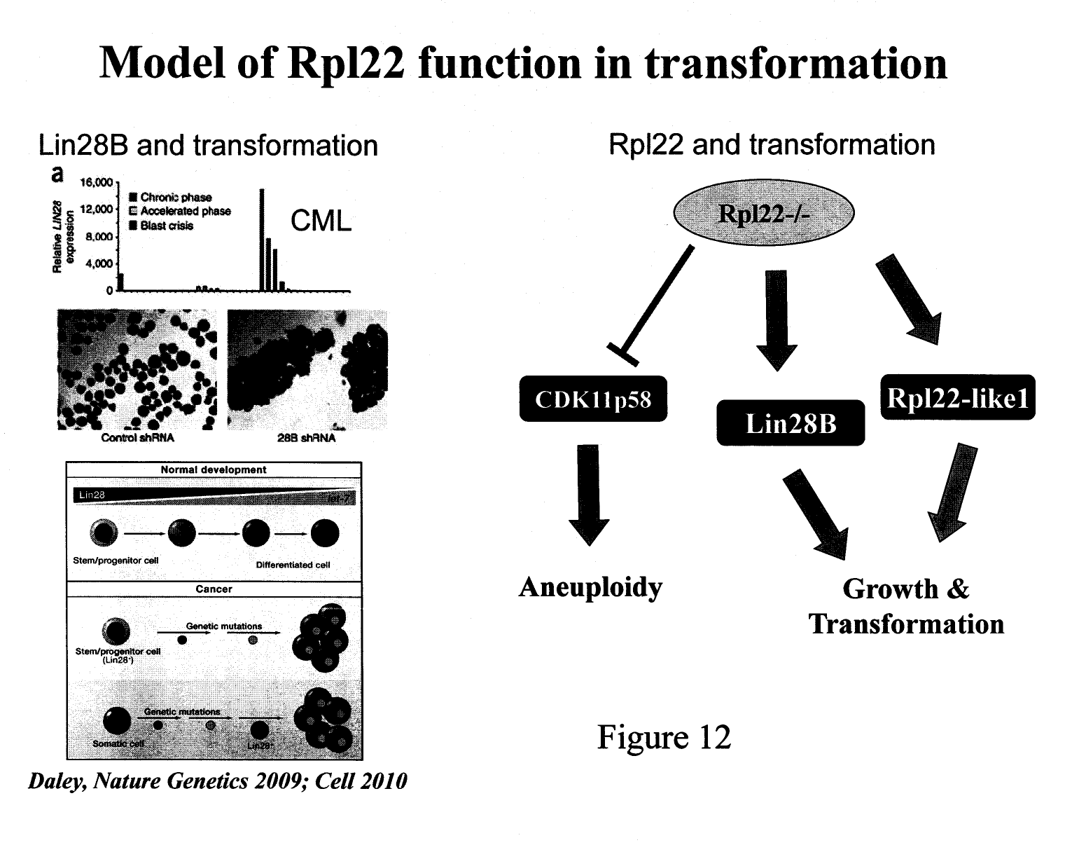

In summary, our analysis reveals that Rpl22 plays a heretofore unsuspected, but important role in regulating development of thymocytes. Indeed, ablation of the Rpl22 gene activates a p53-dependent checkpoint that selectively blocks development of immature precursors of the αβ T lineage. Additionally, mutations or loss in Rpl22 function is associated with an increased propensity for the development of T-ALL as well as an increased risk of relapse after treatment. At present, our data also suggests that the mechanism by which Rpl22 mutations promote the development of cancer involves the disregulation of a minimum of 3 different molecular effectors (FIG. 12). The first is CDK11p58, a protein that is required for accurate chromosome segregation. Accordingly, reduced expression of Rpl22 impairs expression of CDK11p58, resulting in aneuploidy in tumors bearing Rpl22 mutations. Second, Rpl22 inactivation results in induction of Lin28B which is a pluripotency inducing factor that has been observed to be elevated in late-stage, aggressive tumors, and which can promote growth and transformation when overexpression. Finally, Rpl22 mutations also induce the expression of Rpl22-like1, the paralog of Rpl22. Like Lin28B, Rpl22-like1 promotes growth and may assist in driving transformation. Therefore, elevations in Lin28B and Rpl22-like1 may both represent useful and more readily measured biomarkers of aggressive tumors, whose aggressive behavior results from Rpl22 loss.

While certain of the preferred embodiments of the present invention have been described and specifically exemplified above, it is not intended that the invention be limited to such embodiments. It will be apparent to one skilled in the art that various changes and modifications can be made therein without departing from the scope of the present invention, as set forth in the following claims.

What is claimed is:

1. A method for detecting a propensity for developing cancer the method comprising: detecting the presence of a mutation in an Rpl22 coding sequence in a target polynucleotide wherein said mutation alters the function of Rpl22 and when said mutation is present, said patient has an increased risk for developing cancer.

2. The method of claim 1, wherein said mutation is a deletion and said cancer is T-ALL

3. A method as claimed in claim 1, wherein the target nucleic acid is amplified prior to detection.

4. The method of claim 1, wherein the step of detecting the presence of said mutation comprises performing a process selected from the group consisting of detection of specific hybridization, measurement of allele size, restriction fragment length polymorphism analysis, allele-specific hybridization analysis, single base primer extension reaction, and sequencing of an amplified polynucleotide.

5. A method as claimed in claim 1, wherein in the target nucleic acid is DNA.

6. The method of claim 1, wherein nucleic acids comprising said mutation are obtained from an isolated cell of the human subject.

7. A method for identifying agents which modulate the development of cancer:
a) providing cells which lack functional Rpl22 protein;
b) providing cells which express native Rpl22 protein;
c) contacting the cells of steps a) and b) with a test agent and
d) analyzing whether said agent alters an Rpl22-associated parameter in cells contacted in step a) relative to those of step b), thereby identifying agents which modulate the development of cancer.

8. The method of claim 8, wherein said cancer is T-ALL.

9. The method of claim 7, wherein said Rpl22 associated parameter is selected from the group consisting of aberrant translation, aberrant T cell proliferation, aberrant T cell differentiation, increased rate of transformation, altered expression of CDK11p58, altered expression levels of Lin 28, altered expression of Rpl22-Like 1 and increased cellular proliferation.

10. The method of claim 7, wherein said cells are selected from the group consisting of peripheral blood mononuclear cells, T lymphocytes, neurons, MEF cells, cells comprising a mutation in human chromosome 1p36.3-1p36.2, Rpl22^{+/-} cells, Rpl22^{-/-} cells, and T lineage progenitor cells.

11. The method of claim 7, wherein said cells are within an animal in which the Rpl22 locus has been ablated.

12. A method of treating or inhibiting cancer progression in a patient in need thereof comprising administering an effective amount of an agent which restores the function of the Rpl22 gene product, thereby reducing symptoms associated with cancer.

13. A method of diagnosing an increased propensity for relapse of T-ALL comprising;
a) obtaining a biological sample from a patient previously diagnosed with T-ALL;
b) detecting the presence of a mutation in an Rpl22 coding sequence in a target polynucleotide relative to a control wild type sequence lacking said mutation, wherein if said mutation is present, said patient has an increased risk for relapse of T-ALL.

14. The method of claim 13, wherein said mutation is a single nucleotide deletion.

15. The method of claim 14, wherein said deletion is a monoallelic loss of an A.
